(19) United States
(12) Patent Application Publication
(10) Pub. No.: US 2020/0306469 A1
(43) Pub. Date: Oct. 1, 2020

(54) RESPIRATORY MONITOR IN A NONINVASIVE VENTILATOR

(71) Applicant: KONINKLIJKE PHILIPS N.V., Eindhoven (NL)

(72) Inventors: ROBERT ANTHONY ROMANO, PITTSBURGH, PA (US); RICHARD JAMES MCKENZIE, JR., BUTLER, PA (US)

(21) Appl. No.: 16/815,411

(22) Filed: Mar. 11, 2020

Related U.S. Application Data
(60) Provisional application No. 62/825,250, filed on Mar. 28, 2019.

(51) Int. Cl. A61M 16/00

(52) U.S. Cl. CPC A61M 16/0051; A61M 16/024; A61M 2016/0033; A61M 2205/3592; A61M 2230/46; A61M 2205/3584; A61M 2205/18

(57) ABSTRACT

The present patent application discloses a system that includes a pressure generator; one or more sensors configured to generate output signals conveying information related to one or more parameters of a pressurized flow of breathable gas to an airway of the subject and respiration of the subject; and one or more hardware processors configured by machine readable instructions to: determine the one or more parameters of the pressurized flow of breathable gas and the respiration of the subject based on the information in the output signals; determine one or more indices of respiratory impedance for the airway of the subject based on the determined parameters; determine a change in health status of the subject based on the one or more indices of respiratory impedance; and generate an alert indicative of the change in health status for communication to the subject and/or a caregiver of the subject.

### CROSS-REFERENCE TO RELATED APPLICATIONS

This patent application claims the priority benefit under 35 U.S.C. § 119(e) of U.S. Provisional Application No. 62/825,250, filed on Mar. 28, 2019, the contents of which are herein incorporated by reference.

### BACKGROUND

### 1. Field

The present patent application discloses a system and a method for providing pressure therapy to a subject. Specifically, the system and method is configured to determine a change in health status of the subject based on determined one or more indices of respiratory impedance, and generate an alert indicative of the change in health status for communication to the subject and/or a caregiver of the subject.

### 2. Description of the Related Art

A ventilation system or ventilator delivers respiratory/pressure therapy to a patient by delivering a gas to the patient's pulmonary system at a level above ambient pressure during inspiration.

Chronic Obstructive Pulmonary Disease (COPD) is a progressive chronic inflammatory lung disease that causes obstructed airflow from the lungs that predisposes to exacerbations and serious illness. The Global Burden of Disease Study reports a prevalence of 251 million cases of COPD globally in 2016. Worldwide, it is estimated that over 3 million deaths annually are caused by the disease. However, it has been estimated that less than 40% of patients diagnosed with COPD have ever undergone a formal diagnosis of their disease with spirometry. Moreover, spirometry is not able to satisfactorily determine a person's expiratory flow limitation (EFL). Thus, follow-up and continuous monitoring of a person's lung disease is not common. Currently, patients with advanced progression of their COPD are treated with noninvasive ventilation. This therapy is intended to be administered daily/nocturnally. In addition, studies have shown that drastic changes in symptoms are important events in patients with COPD because they contribute to a further decline in lung function, impaired health-related quality of life, socioeconomic burden, and poor prognosis.

Therefore, an improved system and method is provided to overcome the above-discussed problems and disadvantages.

### SUMMARY

Accordingly, one or more aspects of the present patent application relate to a system configured to provide pressure therapy to a subject. The system comprises a pressure generator configured to provide a pressurized flow of breathable gas to an airway of the subject; one or more sensors configured to generate output signals conveying information related to one or more parameters of the pressurized flow of breathable gas and respiration of the subject; and one or more hardware processors operatively connected with the pressure generator and the one or more sensors. The one or more hardware processors are configured by machine readable instructions to: determine the one or more parameters of the pressurized flow of breathable gas and the respiration of the subject based on the information in the output signals; determine one or more indices of respiratory impedance for the airway of the subject based on the determined one or more parameters; determine a change in health status of the subject based on the one or more indices of respiratory impedance; and generate an alert indicative of the change in health status for communication to the subject and/or a caregiver of the subject.

Another aspect of the present patent application relates to a method for providing pressure therapy to a subject. The method is implemented by a computer system that comprises one or more hardware processors executing machine readable instructions that, when executed, perform the method. The method comprises obtaining, from one or more sensors, information related to one or more parameters of a pressurized flow of breathable gas to an airway of the subject and respiration of the subject; determining, using the computer system, the one or more parameters of the pressurized flow of breathable gas and the respiration of the subject based on the obtained information; determining, using the computer system, one or more indices of respiratory impedance for the airway of the subject based on the determined one or more parameters; determining, using the computer system, a change in health status of the subject based on the one or more indices of respiratory impedance; and generating, using the computer system, an alert indicative of the change in health status for communication to the subject and/or a caregiver of the subject.

Yet another aspect of the present patent application relates to a system for providing pressure therapy to a subject. The system comprises a means for providing a pressurized flow of breathable gas to an airway of the subject; a means for generating output signals conveying information related to one or more parameters of the pressurized flow of breathable gas and respiration of the subject; and a means for executing machine-readable instructions with at least one hardware processor, wherein the machine-readable instructions comprise obtaining, from the means generating output signals, information related to one or more parameters of the pressurized flow of breathable gas and the respiration of the subject; determining, using the means for executing, the one or more parameters of the pressurized flow of breathable gas and the respiration of the subject based on the obtained information; determining, using the means for executing, one or more indices of respiratory impedance for the airway of the subject based on the determined one or more parameters; determining, using the means for executing, a change in health status of the subject based on the one or more indices of respiratory impedance; and generating, using the means for executing, an alert indicative of the change in health status for communication to the subject and/or a caregiver of the subject.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a system for providing pressure therapy to a subject in accordance with an embodiment of the present patent application;

FIG. 2 shows another system for providing the pressure therapy to the subject in accordance with an embodiment of the present patent application;

FIG. 3 shows Expiratory Flow Limitation (EFL), which is a condition when an increase in transpulmonary pressure causes no corresponding increase in expiratory flow due to “choke points” in the many bronchial branches in accordance with an embodiment of the present patent application;

FIG. 4 shows an exemplary periodogram in which an event is triggered when a portion (i.e., number of samples) of the spectral data falls within a certain region in accordance with an embodiment of the present patent application;

FIG. 5 shows an exemplary graphical representation of information related to one or more indices of the spectral data for an airway of the subject in accordance with an embodiment of the present patent application;

FIG. 6 shows an exemplary graphical representation of seasonal variability changes and trends in accordance with an embodiment of the present patent application;

FIG. 7 shows an exemplary graphical representation of seasonal variability with a trend in accordance with an embodiment of the present patent application;

FIG. 8 shows an exemplary graphical representation of increasing seasonal variability with time in accordance with an embodiment of the present patent application;

FIG. 9 shows an exemplary graphical representation of increasing variability with time in accordance with an embodiment of the present patent application;

FIG. 10 shows an exemplary graphical representation of changes in the variability over time in accordance with an embodiment of the present patent application;

FIG. 11 shows an exemplary graphical representation of information related to the spectral data for the airway of the subject in accordance with an embodiment of the present patent application; and

FIG. 12 shows a method for providing pressure therapy to the subject in accordance with an embodiment of the present patent application.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

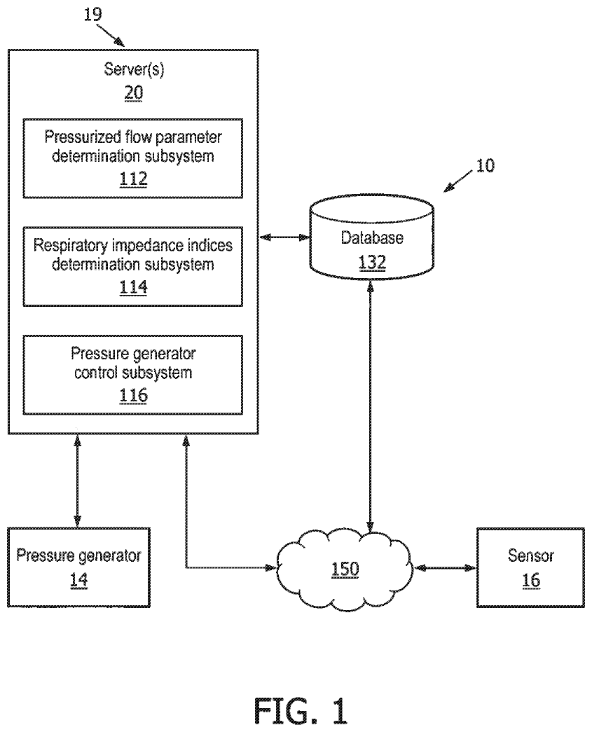

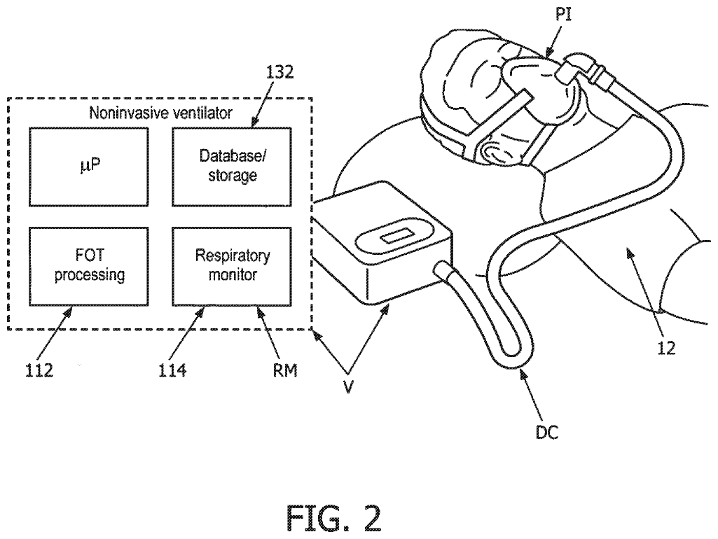

FIGS. 1 and 2 schematically illustrate a system 10 configured to provide pressure therapy to a subject 12. System 10 comprises a pressure generator 14 configured to provide a pressurized flow of breathable gas to an airway of subject 12; one or more sensors 16 configured to generate output signals conveying information related to one or more parameters of the pressurized flow of breathable gas and respiration of subject 12; and one or more hardware processors 20 operatively connected with pressure generator 14 and one or more sensors 16. One or more hardware processors 20 are configured by machine readable instructions to: determine the one or more parameters of the pressurized flow of breathable gas and the respiration of subject 12 based on the information in the output signals; determine one or more indices of respiratory impedance for the airway of subject 12 based on the determined one or more parameters; determine a change in health status of subject 12 based on the one or more indices of respiratory impedance; and generate an alert indicative of the change in health status for communication to subject 12 and/or a caregiver of subject 12. In some embodiments, in this patent application, subject may be interchangeably referred to as a consumer, a user, an individual or a patient.

The Expiratory Flow Limitation (EFL) is a physiological condition where a person's airways lose their elastic recoil due to parenchymal destruction which causes the airways to partially collapse during expiration. A method called Forced Oscillation Technology (FOT) is a noninvasive method used to measure lung mechanics. Studies have determined that a person's expiratory reactance, derived from respiratory impedance, and, therefore. their EFL can be minimized by the application of external Positive Expiratory Pressure (PEP).

The present patent application provides a noninvasive ventilator with an internal ability to deliver precision FOT. The ventilator is configured to continually assess a person's pulmonary mechanics, and automatically apply an appropriate setting of Positive Expiratory Pressure that is able to counterbalance intrinsic PEEP and treat a person's EFL on a breath-by-breath basis. This therapy can be delivered in either the seated or supine positions, allowing for accurate assessments of the EFL under different postural conditions, useful for stratifying patients by the severity of their Chronic Obstructive Pulmonary Disease (COPD) and disease progression. Subsequently, the present patent application provides a means and a method to store the respiratory indices from the Forced Oscillation Technique calculations. Analyses of these stored parameters are useful for tracking and determining changes in a person's pulmonary mechanics. This information can also be used by a clinician to pre-emptively treat and adjust therapy for this patient.

Respiratory Monitoring System (RMS) of the present patent application is configured to continuously or intermittently track a subject's pulmonary mechanics while the patient is being ventilated in a home or clinical setting and alert the patient's care team to the pulmonary health changes via an automated informational message intended to be an early warning and can be sent through a Patient and Care Management System. The methods within the Respiratory Monitoring System use advanced statistical analytics, as well as, Machine Learning and Artificial Intelligence to determine advance early warning messages.

As shown in FIGS. 1 and 2, system 10 includes a ventilator V configured for delivering bi-level or CPAP pressure support. In some embodiments, ventilator V is a noninvasive ventilator. In some embodiments, ventilator V is a Continuous positive airway pressure (CPAP) ventilator. In some embodiments, ventilator V is a bi-level ventilator. In some embodiments, ventilator V generally includes pressure generator 14 configured to provide breathing gas to subject 12; a patient or delivery circuit DC operatively coupled to pressure generator 14 to deliver the flow of breathing gas to subject 12; and a patient interface PI operatively coupled to the patient/delivery circuit to communicate the flow of breathing gas to the airway of subject 12.

In some embodiments, pressure generator 14 may be configured to provide the pressurized flow of breathable gas for delivery to the airway of subject 12, e.g., via an output of pressure generator 14, and/or via delivery circuit DC. In some embodiments, pressure generator 14 may be configured to adjust one or more of pressure levels, flow, humidity, velocity, acceleration, and/or other parameters of the pressurized flow of breathable gas, e.g., in substantial synchronization with the breathing cycle of the patient.

In some embodiments, the pressurized flow of breathable gas is delivered from pressure generator 14 to the airway of subject 12 via patient/delivery circuit DC. In some embodiments, delivery circuit DC may include a conduit and/or the patient interface. Delivery circuit DC may sometimes be referred to as patient interface PI. The conduit may include a flexible length of hose, or other conduit, either in single-limb or dual-limb configuration that places the patient interface in fluid communication with pressure generator 14. The conduit forms a flow/fluid path through which the pressurized flow of breathable gas is communicated between patient interface PI and pressure generator 14.

In some embodiments, patient interface PI may be configured to deliver the pressurized flow of breathable gas to the airway of subject 12. As such, patient interface PI may include any appliance/device suitable for this function. In some embodiments, pressure generator 14 is a dedicated ventilation device and patient interface PI is configured to be removably coupled with another interface being used to deliver respiratory therapy to subject 12. For example, patient interface PI may be configured to engage with and/or be inserted into an endotracheal tube, a tracheotomy portal, and/or other interface appliances/devices. In some embodiments, patient interface PI may be configured to engage the airway of the patient without an intervening device. In some embodiments, patient interface PI may include one or more of an endotracheal tube, a nasal cannula, a tracheotomy tube, a nasal mask, a nasal/oral mask, a full-face mask, a total facemask, and/or other interface devices that communicate a flow of gas with an airway of subject 12. The present patent application is not limited to these examples, and contemplates delivery of the pressurized flow of breathable gas to the patient using any subject interface.

In some embodiments, one or more sensors 16 are configured to generate output signals conveying information related to one or more parameters of the pressurized flow of breathable gas and the respiration of subject 12. As another example, the information may be obtained from one or more monitoring devices (e.g., airway flow monitoring device, airway pressure monitoring device, or other airway monitoring devices). In some embodiments, one or more monitoring devices and associated sensors 16 may be configured to monitor flow at the airway opening. In some embodiments, one or more monitoring devices and associated sensors 16 may be configured to monitor pressure at the airway opening. These monitoring devices may include one or more sensors 16, such as pressure sensors, pressure transducers, flow rate sensors, or other sensors. Sensors 16 may, for instance, be configured to obtain information of the patient (e.g., airway pressure, airway flow, or any other airway parameters) or other information related to the patient's airways.

In some embodiments, each of one or more sensors 16 include a transmitter for sending signals and a receiver for receiving the signals. In some embodiments, one or more sensors 16 are configured to communicate wirelessly with computer system 19. As shown in FIG. 1, in some embodiments, sensor 16 is configured to be operatively connected with computer system 19 and/or one or more physical processors 20 of computer system 19. In some embodiments, one or more sensors 16 are configured to communicate with ventilator V. In some embodiments, one or more sensors 16 are in communication with a database 132. In some embodiments, the information related to one or more parameters of the pressurized flow of breathable gas and the respiration of subject may be obtained from the database 132 that is being updated in real-time by one or more sensors 16. In some embodiments, one or more sensors 16 are in fluid communication with breathing or patient passage/circuit/tubing/conduit of ventilator V.

In one scenario, a monitoring device may obtain information (e.g., based on information from one or more sensors 16), and provide information to computer system 19 (e.g., comprising server 20) over a network (e.g., network 150) for processing. In another scenario, upon obtaining the information, the monitoring device may process the obtained information, and provide processed information to computer system 19 over a network (e.g., network 150). In yet another scenario, the monitoring device may automatically provide information (e.g., obtained or processed) to computer system 19 (e.g., comprising server 20). In some embodiments, sensors 16 may be placed close to the mouth of the patient and/or at the ventilator outlet or other locations, with appropriate compensation algorithms to estimate the corresponding airflow and airway pressure in proximity of the patient's mouth. In some embodiments, server 20 includes one or more physical/hardware processors 20. In FIG. 1, database 132 is shown as a separate entity, but, in some embodiments, database 132 could be part of computer system 19.

In some embodiments, ventilator V (e.g., noninvasive, bi-level or CPAP) as shown in FIGS. 1 and 2 is configured to internally deliver the Forced Oscillation Technique (FOT). The FOT works by comparing the phase shift of the small amplitude oscillations on the airway flow signal/information to the oscillations on the airway pressure signal/information. In a healthy unobstructed lung, these two signals/information arrive at the same time to sensors 16. However, whenever there is a pulmonary obstruction or change in the inertial properties of the lung, then there is an offset in the arrival time between these two signals/information (i.e., the airway flow signal and the airway pressure signal).

Airway impedance can be deduced by the mechanical response to these small time varying changes. For example, an oscillatory flow and pressure delivered to the patient's pulmonary system are used to measure the respiratory/pulmonary impedance. The impedance can mathematically be further broken down into two components, respiratory/pulmonary resistance and respiratory/pulmonary reactance. The respiratory/pulmonary impedance measurements are then further broken down into inspiration phase and expiration phase.

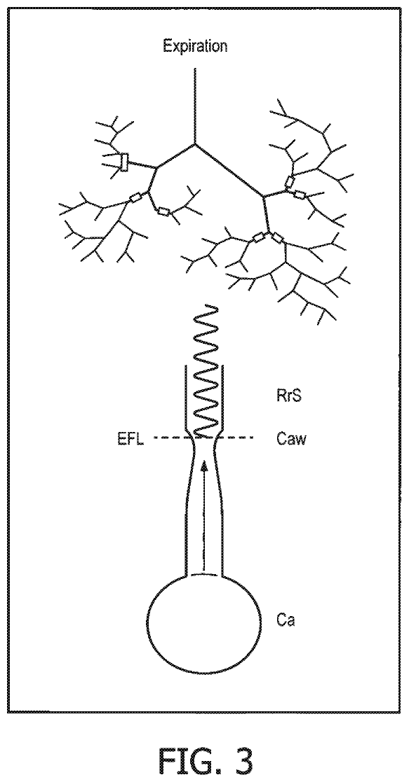

The respiratory/pulmonary reactance, which is the reactance or imaginary component of the respiratory/pulmonary impedance is a useful indicator for the level of the expiratory flow limitation (EFL) that is present. The resistive component is dominant in patients that have airway restrictions such as asthmatics or those with (e.g., lower) airway obstructions. The expiratory reactance component has been shown to correlate to a person's degree of Expiratory Flow Limitation (EFL), a condition when an increase in transpulmonary pressure causes no corresponding increase in expiratory flow due to “choke points” in the many bronchial branches (see FIG. 3) which is the hallmark of Chronic Obstructive Pulmonary Disease (COPD). The consequence of “choke points” lead to an increase in work of breathing and dyspnea or shortness of breath. In some embodiments, respiratory/pulmonary reactance may be determined using the information related to one or more parameters of the pressurized flow of breathable gas and the respiration of the subject.

In some embodiments, system 10 includes one or more hardware processors 20 operatively connected with pressure generator 14 and one or more sensors 16.

As shown in FIG. 1, system 10 may comprise server 20 (or multiple servers 20). In some embodiments, server 20 comprises pressurized flow parameter determination subsystem 112, respiratory impedance indices determination subsystem 114, pressure generator control subsystem 116 or other components or subsystems.

As will be clear from the discussions above and below, in some embodiments, system 10 includes computer system 19 that has one or more physical/hardware processors 20 programmed with computer program/machine readable instructions that, when executed cause computer system 19 to obtain information or data from one or more sensors 16. In some embodiments, computer system 19 may also be referred to as means 19 for executing machine readable instructions with at least one hardware processor 20.

In some embodiments, pressurized flow parameter determination subsystem 112 is configured to determine the one or more parameters of the pressurized flow of breathable gas and the respiration of the subject based on the information in the output signals generated by one or more sensors 16. In some embodiments, the one or more parameters of the pressurized flow of breathable gas and the respiration of subject 12 may include respiration rate, tidal volume, expiratory tidal volume, respiratory airway pressure, respiratory airway flow, or other respiratory airway parameters. In some embodiments, the one or more parameters of the pressurized flow of breathable gas and the respiration of subject 12 may include inspiratory:expiratory (I:E) ratio, total circuit leak, mask or interface leak, intentional leak, unintentional leak, Inspiratory positive airway pressure (IPAP), Expiratory positive airway pressure (EPAP), etc. In some embodiments, pressurized flow parameter determination subsystem 112 is configured to receive or obtain information about flow of air (into and out of the respiratory system) and information about the airway opening pressure (measured, for instance, at a Y-piece of the ventilator) as the inputs.

In some embodiments, respiratory impedance indices determination subsystem 114 is configured to determine one or more indices of respiratory impedance for the airway of the subject based on the determined parameters. In some embodiments, the one or more indices of respiratory impedance for the airway of subject 12 may include respiratory resistance value, respiratory impedance value, respiratory reactance value, or inspiratory reactance value. In some embodiments, the one or more indices of respiratory impedance for the airway of subject 12 may include percentage of Flow Limited Breaths, lung impedance value, total system airway resistance value, day-by-day changes of lung mechanical impedance value, or day-by-day changes of breathing pattern. In some embodiments, the one or more indices of respiratory impedance for the airway of subject 12 may include percentage/count of valid breaths over a time period or a set number of breaths, percentage/count of non-flow limited breaths over a time period or a set number of breaths, etc. In some embodiments, one or more hardware processors 20 are configured such that the expiratory reactance value is a total airway expiratory reactance value, and wherein the total airway of the subject comprises a mouth, nasal passages, sinuses, pharynx, trachea, and lungs of subject 12.

In some embodiments, one or more hardware processors 20 are configured such that the determined one or more indices of respiratory impedance comprise a respiratory resistance value, a respiratory impedance value, and a respiratory reactance value. In some embodiments, the expiratory reactance value is determined based on the respiratory impedance value.

In some embodiments, one or more hardware processors 20 are configured such that the respiratory resistance value, the respiratory impedance value and the respiratory reactance value are determined for individual breaths in an ongoing manner during the ventilation therapy.

In some embodiments, one or more hardware processors 20 are configured such that determining the determined one or more indices of respiratory impedance for subject 12 based on the determined parameters comprises determining a breath to breath change in the expiratory reactance value. In some embodiments, one or more hardware processors 20 are configured to determine the change in health status of subject 12 based on the breath to breath change in the expiratory reactance value to provide the ventilation therapy to subject 12. In addition to breath by breath change, in some embodiments, it is possible consider a group of breaths or some time period. In some embodiments, one or more hardware processors 20 are further configured to determine a threshold for the breath to breath change in the expiratory reactance value, and generate the alert indicative of the change in health status responsive to a breach of the threshold by the breath to breath change in the expiratory reactance value.

In some embodiments, one or more hardware processors 20 are further configured to determine the threshold for the breath to breath change in the expiratory reactance value based on a seasonality or a periodicity of the breath to breath change in the expiratory reactance value. In some embodiments, one or more hardware processors 20 are further configured to determine the threshold for the breath to breath change in the expiratory reactance value based on a seasonality of the breath to breath change in the expiratory reactance value. In some embodiments, one or more hardware processors 20 are further configured to determine the threshold for the breath to breath change in the expiratory reactance value based on a periodicity of the breath to breath change in the expiratory reactance value.

In some embodiments, one or more hardware processors 20 are further configured to control pressure generator 14 to adjust the pressurized flow of breathable gas based on the generated alert and the determined one or more indices of respiratory impedance to provide the pressure therapy to subject 12.

In some embodiments, system 10 includes using noninvasive ventilator V to track pulmonary status using indices of respiratory impedance. In some embodiments, a subject's pulmonary mechanics are collected from one or more sensors 16 in ventilator V (e.g., impedance, reactance, resistance, respiration rate, tidal volume, etc.) and are compared to values that are set or determined within ventilator V or an external patient management system.

The respiratory monitor RM within ventilator V then derives, stores and analyses the respiratory impedance indices, as well as, other relevant information from ventilator's sensors 16. In some embodiments, respiratory monitor RM is configured to compare the respiratory impedance indices against baseline parameters of subject 12. In some embodiments, respiratory monitor RM of ventilator V includes respiratory impedance indices determination subsystem 114.

In some embodiments, the comparison of the respiratory impedance indices against the baseline parameters of the subject can be done by single points of data. In some embodiments, the comparison of the respiratory impedance indices against the baseline parameters of the subject can be done by a time interval trend. In some embodiments, the comparison of the respiratory impedance indices against the baseline parameters of the subject can be done by a variability of one or many indices within a time period.

In some embodiments, respiratory monitor RM or respiratory impedance indices determination subsystem 114 is also configured to determine a deviation of these respiratory impedance indices, either singularly or in plurality from baseline parameters, in order to assess the pulmonary status of subject 12. For instance, in some embodiments, it has been shown that changes in the difference between the mean reactance in the expiratory phase and the mean reactance in the inspiratory phase were significantly larger in a group of COPD patients that may ultimately experience negative health consequences. As further example, in some embodiments, a decrease in negativity of the mean expiratory reactance is indicative of improving pulmonary health and a reduction of the expiratory flow limitation (EFL).

In some embodiments, pressure generator control subsystem 116 is configured to control pressure generator 14 to adjust the pressurized flow of breathable gas based on the determined one or more indices of respiratory impedance to provide the ventilation therapy to subject 12. In some embodiments, pressure generator control subsystem 116 is configured to control pressure generator 14 to adjust one or more of pressure levels, flow, humidity, velocity, acceleration, and/or other parameters of the pressurized flow of breathable gas based on the one or more indices of respiratory impedance to provide the ventilation therapy to subject 12.

In some embodiments, pressure generator control subsystem 116 is configured to control pressure generator 14 to adjust the pressurized flow of breathable gas based on a comparison of the respiratory impedance indices against the baseline parameters of the subject to provide the ventilation therapy to subject 12.

In some embodiments, pressure generator control subsystem 116 is configured to control pressure generator 14 to adjust the pressurized flow of breathable gas based on a comparison of the respiratory impedance indices against values that are set or determined within ventilator V or an external patient management system. In some embodiments, these values may include 1) preset default (predetermined) values based on the type of patient and the stage of their disease; 2) a subject specific established baseline derived from a baseline period of time or patient current or past health status; 3) configured values through a Ventilator User Interface; or 4) limits set within an external care management system (e.g., Care Orchestrator or Clinical or Hospital system).

In some embodiments, the rule based database is constructed of the following: 1) day by day values, 2) trended data (i.e., over a period of time), 3) Time series analysis (i.e., identified patterns in time series data; created by machine learning algorithms, which use a multiplicity of methods that include, but not limited to: Cross-Periodogram, Cross-Density, Quadrature-Density, Cross-Amplitude or Squared Coherency), and/or 4) advanced statistical analytics, running average SD, or other variability indices.

In some embodiments, the comparison of the respiratory impedance indices against the baseline parameters of the subject or against values that are set or determined within ventilator V or an external patient management system to provide the ventilation therapy to subject 12 is performed by respiratory impedance indices determination subsystem 114. In some embodiments, the comparison of the respiratory impedance indices against the baseline parameters of the subject or against values that are set or determined within ventilator V or an external patient management system to provide the ventilation therapy to subject 12 is performed by pressure generator control subsystem 116.

In some embodiments, a subsystem of system 10 is configured to continuously obtain subsequent information related to one or more parameters of the pressurized flow of breathable gas and the respiration of subject 12. As an example, the subsequent information may comprise additional information corresponding to a subsequent time (after a time corresponding to information that was used to control pressure regulator 14). The subsequent information may be utilized to further update or modify the baseline parameters of subject 12 or values that are set or determined within ventilator V/CPAP or an external patient management system (e.g., new information may be used to dynamically update or modify the baseline parameters of subject 12 or values that are set or determined within ventilator V or an external patient management system), etc. For example, the subsequent information may also be configured to provide further input to determine the one or more indices of respiratory impedance.

In some embodiments, a subsystem of system 10 may be configured to determine the one or more indices of respiratory impedance and/or to control pressure generator 14 to adjust the pressurized flow of breathable gas in accordance with a recursively refined profile (e.g., refined through recursive application of profile refinement algorithms) based on previously collected or subsequent information related to one or more parameters of the pressurized flow of breathable gas and the respiration of subject 12.

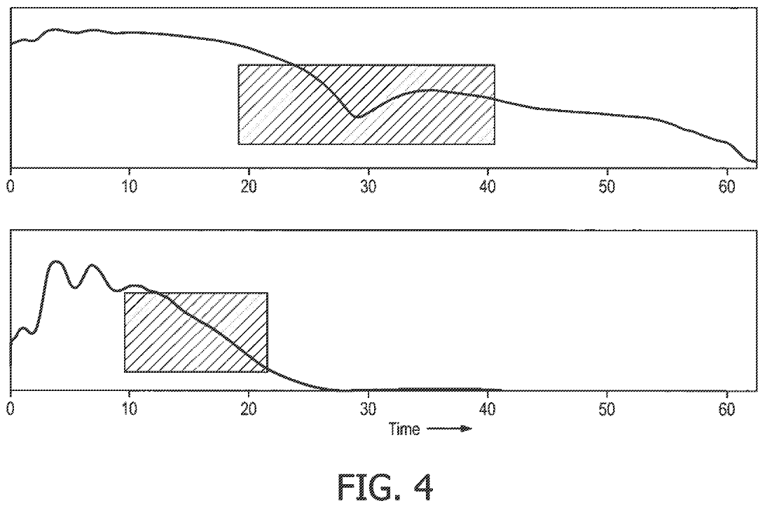

FIG. 4 shows an exemplary periodogram in which an event is triggered when a portion (i.e., number of samples) of the spectral data/information falls within or outside a certain region in accordance with an embodiment of the present patent application. The top graph of FIG. 4 shows the amplitude vs frequency waveform. Frequency is on the X-axis of the graph and is measured in Hertz (Hz). The bottom graph of FIG. 4 shows the power vs frequency waveform. Frequency is shown on the X-axis of all the graphs in FIG. 4 and is measured in Hertz (Hz).

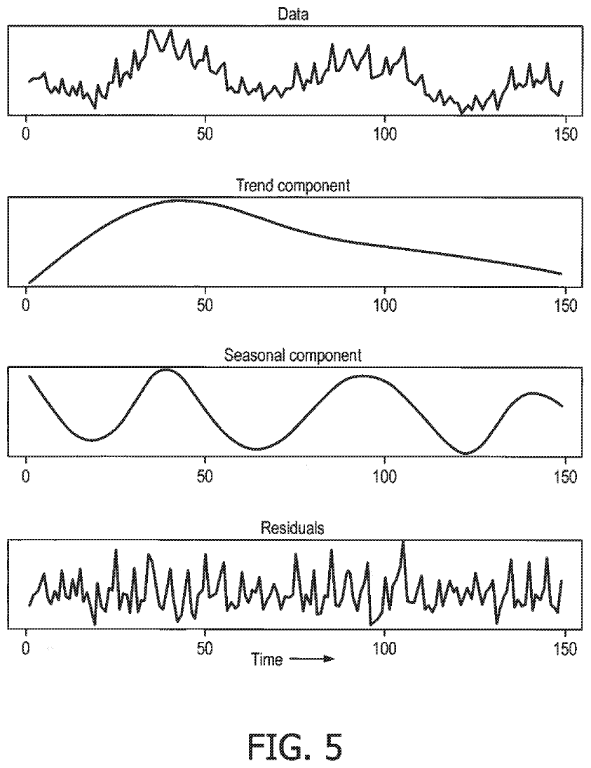

FIG. 5 shows exemplary waveforms for data, trend component, seasonal component, and residuals in accordance with an embodiment of the present patent application. The graphs relate to a person's stable or changing health status and or their lung parameters or factors in their pulmonary/respiratory indexes. The topmost graph of FIG. 5 shows the data waveform. The second graph from the top of FIG. 5 shows the trend component waveform. The third graph from the top of FIG. 5 shows the seasonal component waveform. The bottommost graph of FIG. 5 shows the residuals waveform. Time is shown on the X-axis of all the graphs shown in FIG. 5 and is measured in seconds. In some embodiments, time on the X-axis could be measured in minutes, hours, days, weeks, months, etc.

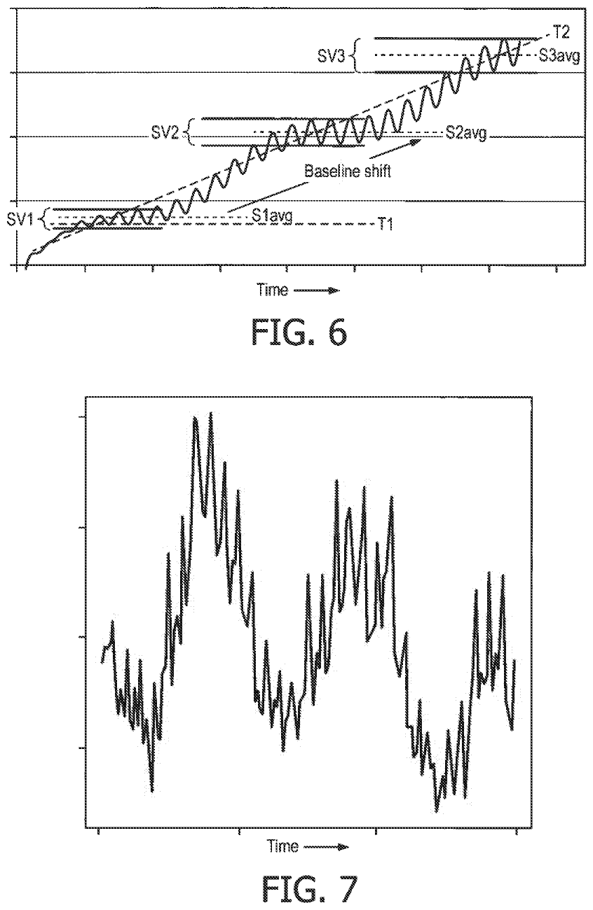

FIG. 6 shows an exemplary graphical representation of seasonal variability changes and trends in accordance with an embodiment of the present patent application. In some embodiments, referring to FIG. 6, “seasonal” variability changes are represented by SV1, SV2 & SV3; Trends are represented by T1 and T2.

In some embodiments, “seasonal,” as used herein, can correspond to: calendar season changes, weather patterns, increased airborne particulate such as pollen and pollution, cold/flu season or other environmental conditions that affect a human's health during certain times. In some embodiments, “seasonal,” as used herein is a statistical term. Time is shown on the X-axis of the graph and is measured in seconds. In some embodiments, time on the X-axis could be measured in minutes, hours, days, weeks, months, etc.

In some embodiments, data/information can change with periodicity, that is, at any given moment be at one value then increase or decrease at another moment with wide variances (“seasonality”) (e.g., see SV1, SV2, SV3 in FIG. 6) and be considered acceptable physiologically as within normality or acceptability. But a second simultaneous periodicity (e.g., see S1avg, S2avg, S3avg in FIG. 6) can occur where there is a continually increase or decrease in a running average or the standard deviation that can be described as trending out of normal and be considered at risk. This time series data is often described in terms of two basic classes of components, trends and seasonality (or possibly cyclic). In some embodiments, classical decomposition

Xt=mt+st+Yt,

where:

mt=trend component (relatively smooth pattern that is slowly changing in time);

st=seasonal component (pattern that appears in regular intervals); and

Yt=residuals or random noise component

In the case of a subject's respiratory parameters, each property of a breath can differ from the next, but over an average, within a given time period, be within an average range and possess a linear correlation (e.g., see T1 in FIG. 6) with time that is near zero. However, a subject's pulmonary status can change due to some perturbation either to some benefit from receiving medication or improving health. Or, on the other hand, a subject's pulmonary status can change detrimentally due to illness. In either case, the periodicity of the respiratory trend data can change (e.g., see T2 in FIG. 6). To compound the difficulties in detecting these systematic changes is that there is a likelihood of noise and erroneous data within the “good” data/information. In some embodiments, various forms of filtering can be applied to remove such outliers, but as often is the case, the data/information becomes either skewed or a time delay is introduced. Also, when considering physiological data/information, a second “seasonal” component arises, where the changes in the first seasonal component become more pronounced or reduced. That is to say, the otherwise normal breath by breath or day by day variance may change (e.g., see SV1, SV2, SV3 in FIG. 6). Alternatively, there can be a shift in baseline of the average variability such as S1avg going to S2avg (e.g., see FIG. 6) which could correspond to a change in physiological health.

FIG. 7 shows an exemplary graphical representation of seasonal variability with a trend in accordance with an embodiment of the present patent application. Example of this, there are more patients that have increased illness in the winter periods. Patients may become more ill over the time period as seasons starts slowly, then maximizes and then takes off like a bell curve. Time is shown on the X-axis of the graph and is measured in seconds. In some embodiments, time on the X-axis could be measured in minutes, hours, days, weeks, months, etc.

In some embodiments, when a subject's data exceed the range and rules that are prescribed, then a designed protocol will be invoked that may issue an alert/notification to a monitoring clinical provider or team requiring an action to acknowledge and respond.

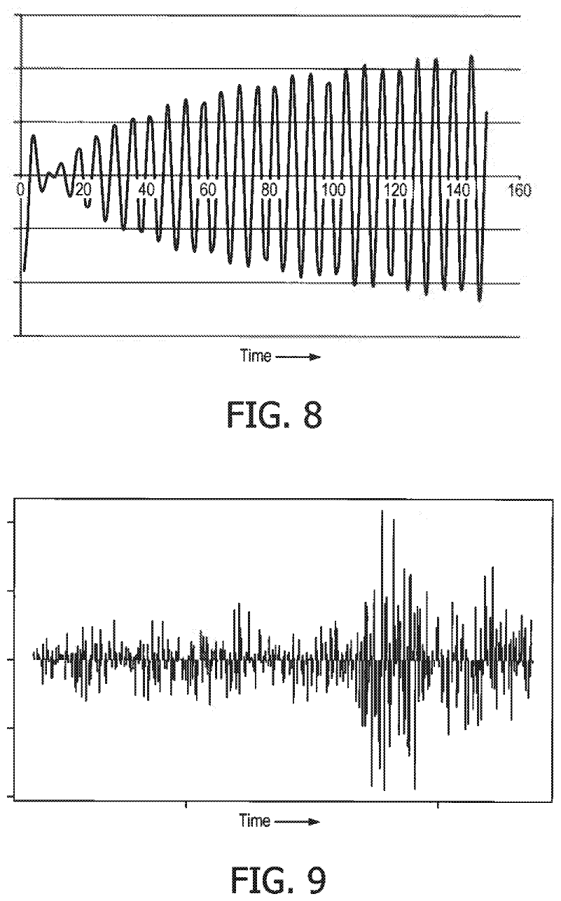

FIG. 8 shows an exemplary graphical representation of increasing seasonal variability with time in accordance with an embodiment of the present patent application. In some embodiments, the average value remains relatively constant while the Standard Deviation or other indices of variability are increasing. Time is shown on the X-axis of the graph and is measured in seconds. In some embodiments, time on the X-axis could be measured in minutes, hours, days, weeks, months, etc.

FIG. 9 shows an exemplary graphical representation of increasing variability with time in accordance with an embodiment of the present patent application. Time is shown on the X-axis of the graph and is measured in seconds. In some embodiments, time on the X-axis could be measured in minutes, hours, days, weeks, months, etc.

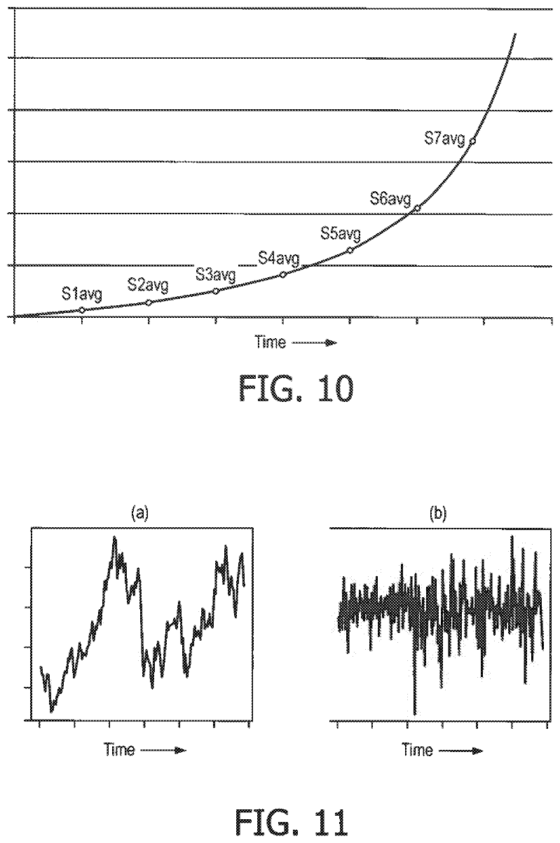

FIG. 10 shows an exemplary graphical representation of changes in the variability over time in accordance with an embodiment of the present patent application. In some embodiments, an event flag is triggered when data/information exceeds threshold. [Time is shown on the X-axis of the graph and is measured in seconds. In some embodiments, time on the X-axis could be measured in minutes, hours, days, weeks, months, etc.

In some embodiments, another example of advanced analytics that could be used by this the Respiratory Monitoring System (RMS) is referred to differencing; where the difference between two adjacent data point is considered (e.g., see FIG. 11). In some embodiments, if the difference or a number of difference points within a time period exceeds a set of limits then an informational alert is issued.

FIG. 11 shows exemplary graphical representations information related to one or more parameters of a pressurized flow of breathable gas and respiration of subject 12. In some embodiments, referring to FIG. 11, the first difference of a time series is the series of changes from one period to the next. In some embodiments, if Yt denotes the value of the time series Y at period t, then the first difference of Y at period t is equal to Yt-Yt-1. Time is shown on the X-axis of both the left and the right side graphs of FIG. 11 and is measured in seconds. In some embodiments, time on the X-axis could be measured in minutes, hours, days, weeks, months, etc.

In some embodiments, all the graphs shown in FIG. 5-11 may have frequency (measured in Hertz (Hz)), instead of time (measured in seconds), on the X-axis. In some embodiments, time on the X-axis could be measured in minutes, hours, months, etc. In some embodiments, numerics and units of the Y axes in all the graphs shown in FIG. 4-11 are not shown because the data graphed could be any of the parameters listed in the present patent application. All the graphs shown in FIG. 4-11 are intended to be examples.

In some embodiments, for example, trends, variability or absolute values to any of the following features would trigger an event flag that could be monitored by the ventilator monitor subsystem (or external patient care management system). In some embodiments, the features include Expiratory Reactance, Percentage of Flow Limited Breaths, Lung Impedance, Total System Airway Resistance, day-by-day changes of lung mechanical impedance, or day-by-day changes of breathing pattern.

In some embodiments, one or more hardware processors 20 are further configured to determine a change in health status of subject 12 based on the one or more indices of respiratory impedance, and generate an alert indicative of the change in health status for communication to the subject and/or a caregiver of subject 12.

In some embodiments, the alert requires the clinical team/caregiver to intervene with patient 12 or to change the settings of ventilator V. In some embodiments, alternatively, ventilator V is configured to automatically adjust its settings to resolve the alert condition. In some embodiments, the alert requires the clinical team to reevaluate if patient 12 is on the right therapy or medical device to consider alternative or better treatment options.

In some embodiments, the alert instructs patient 12 to receive or schedule medical attention. In some embodiments, the alert instructs patient 12 to be further diagnosed with a professional sleep analysis. In some embodiments, the alert instructs patient 12 to begin taking prescribed medicine (emergency medicine) for use before seeking medical attention. In some embodiments, the alert instructs patient 12 to consult with a medical professional on lifestyle and diets changes to lessen the impact of Obstructive Sleep Apnea (OSA). In some embodiments, the alert instructs patient 12 to consider other forms of therapy or intervention to lessen the effects of the OSA.

In some embodiments, the alert validates or retests the patient's pulmonary condition to validate alert conditions. In some embodiments, the alert requires patient 12 undergo further clinical testing to determine other negative conditions. In some embodiments, the alert considers postponing facility discharge instructions.

In some embodiments, management of an Obstructive Sleep Index via Inspiratory Reactance and/or Resistance values is enabled during mechanical ventilation. In some embodiments, patient's physiological acceptable range of values is configured, once the measurements exceed the predetermined range, then the care system requires monitoring clinical individuals to acknowledge and react to a) changes in Inspiratory Resistance values, b) changes in Inspiratory Reactance values, i.e., 90 percentile of the weighted resistance and reactance values.

In some embodiments, ventilator V and/or a medical information management system are configured to issue a health change state message derived from the detection monitoring. In some embodiments, the informational alert generated can be displayed locally on ventilator V, stored on the device, or transmitted remotely to a medical information system/patient health record or directly to subject 12.

In some embodiments, computer system 19 is configured to notify a clinician of the determined change in health status of subject 12 based on the one or more indices of respiratory impedance. In some embodiments, when the change in health status of subject 12 indicates a patient needs medical attention within the specified time period, computer system 19 is configured to generate audio and/or visuals alerts and/or messages notifying clinicians thereof. It is contemplated that such a message can be provided to the clinicians via communication network 150. In some embodiments, computer system 19 is also configured to notify only (and all) medical specialists needed for the case. In some embodiments, the alert requires the clinical team to intervene with the patient or to change the settings of ventilator V. In some embodiments, alternatively, ventilator V can automatically adjust its settings to resolve the alert condition.

In some embodiments, the alert requires the clinical team to instruct the patient to receive or schedule medical attention. In some embodiments, the alert requires the clinical team to instruct the patient to begin taking prescribed medicine (e.g., emergency medicine) for use before seeking medical attention. In some embodiments, the alert requires the clinical team to validate or retest the patient's pulmonary condition to validate alert conditions. In some embodiments, the alert requires the clinical team to have the patient undergo further clinical testing to determine other negative conditions. In some embodiments, the alert requires the clinical team to consider postponing facility discharge instructions

In some embodiments, system 10 is configured to manage an Obstructive Sleep Index via inspiratory reactance value and/or respiratory/pulmonary resistance value during mechanical ventilation. In some embodiments, a patient's physiological acceptable range of values is configured, once the measurements exceed the range, then the care system requires monitoring clinical individuals to acknowledge and react to a) changes in inspiratory resistance value, or b) changes in inspiratory reactance value, 90 percentile of the weighted resistance and reactance.

In some embodiments, the alert requires the clinical team to intervene with the patient or to change the settings of ventilator V—or ventilator V can automatically adjust its settings to resolve the alarm condition. In some embodiments, the alert requires the clinical team to instruct the patient to be further diagnosed with a professional analysis. In some embodiments, the alert requires the clinical team to reevaluate if the patient is on the right therapy or medical device or to consider alternative or better treatment options. In some embodiments, the alert requires the clinical team to have the patient consult with medical professional on lifestyle and diets changes to lessen the impact of COPD. In some embodiments, the alert requires the clinical team to consider other forms of therapy or intervention to lessen the effects of COPD.

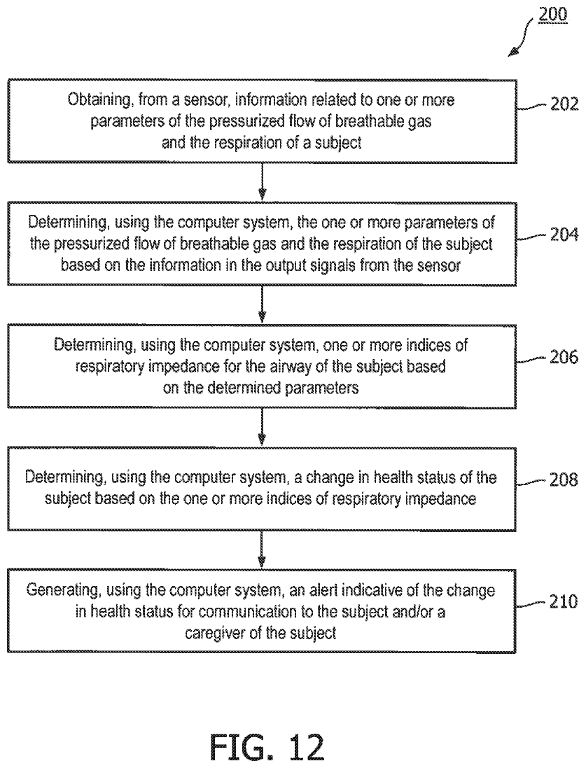

Referring to FIG. 12, a method 200 for providing pressure therapy to subject 12 is provided. Method 200 is implemented by computer system 19 that comprises one or more physical/hardware processors 20 executing computer program/machine readable instructions that, when executed, perform method 200. In some embodiments, method 200 comprises obtaining, from one or more sensors 16, information related to one or more parameters of a pressurized flow of breathable gas and respiration of subject 12 at procedure 202; determining, using computer system 19, the one or more parameters of the pressurized flow of breathable gas and the respiration of the subject based on the information in the output signals generated from one or more sensors 16 at procedure 204; determining, using computer system 19, one or more indices of respiratory impedance for the airway of subject 12 based on the determined one or more parameters at procedure 206; determining, using computer system 19, a change in health status of the subject based on the one or more indices of respiratory impedance at procedure 208; and generating, using computer system 19, an alert indicative of the change in health status for communication to subject 12 and/or a caregiver of subject 12 at procedure 210.

In some embodiments, the one or more indices of respiratory impedance comprise the respiratory resistance value, the respiratory impedance value, and the respiratory reactance value. In some embodiments, method 200 further comprises determining the expiratory reactance value based on the respiratory impedance value. In some embodiments, method 200 further comprises determining a breath to breath change in the expiratory reactance value and determining the change in health status of subject 12 based on the breath to breath change in the expiratory reactance value to provide the ventilation therapy to subject 12.

In some embodiments, method 200 comprises determining a threshold for the breath to breath change in the expiratory reactance value, and generating the alert indicative of the change in health status responsive to a breach of the threshold by the breath to breath change in the expiratory reactance value. In some embodiments, method 200 comprises determining the threshold for the breath to breath change in the expiratory reactance value based on a seasonality or a periodicity of the breath to breath change in the expiratory reactance value.

In some embodiments, method 200 also comprises determining a change in health status of the subject based on the one or more indices of respiratory impedance, and generate an alert indicative of the change in health status for communication to the subject and/or a caregiver of subject 12.

In some embodiments, the various computers and subsystems illustrated in FIGS. 1 and 2 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the servers. As such, the processors may include one or more of a digital processor, an analog processor, or a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112, 114, 116 or other subsystems. The processors may be programmed to execute computer program instructions by software; hardware; firmware; some combination of software, hardware, or firmware; and/or other mechanisms for configuring processing capabilities on the processors. In some embodiments, hardware processors may be interchangeably referred to as physical processors. In some embodiments, machine readable instructions may be interchangeably referred to as computer program instructions.

It should be appreciated that the description of the functionality provided by the different subsystems 112-116 described herein is for illustrative purposes, and is not intended to be limiting, as any of subsystems 112-116 may provide more or less functionality than is described. For example, one or more of subsystems 112-116 may be eliminated, and some or all of its functionality may be provided by other ones of subsystems 112-116. As another example, additional subsystems may be programmed to perform some or all of the functionality attributed herein to one of subsystems 112-116.

It should be appreciated that the different subsystems 112-116 performing the operations illustrated in FIG. 1 may reside in the ventilator itself. In other embodiments, the different subsystems 112-116 performing the operations illustrated in FIG. 1 may reside in an independent monitoring device.

In some embodiments, user interface may be configured to provide an interface between system and a user (e.g., a patient or a caregiver, etc.) through which the user can provide information to and receive information from system 10. This enables data, results, and/or instructions and any other communicable items, collectively referred to as “information,” to be communicated between the user and system 10. Examples of interface devices suitable for inclusion in user interface include a keypad, buttons, switches, a keyboard, knobs, levers, a display screen, a touch screen, speakers, a microphone, an indicator light, an audible alarm, and a printer.

Information may be provided to the patient by the user interface in the form of auditory signals, visual signals, tactile signals, and/or other sensory signals. It is to be understood that other communication techniques, either hard-wired or wireless, are also contemplated herein as the user interface. For example, in one embodiment, the user interface may be integrated with a removable storage interface provided by electronic storage 132. In this example, information is loaded into system 10 from removable storage (e.g., a smart card, a flash drive, a removable disk, etc.) that enables the user(s) to customize system 10. Other exemplary input devices and techniques adapted for use with system 10 as user interface include, but are not limited to, an RS-232 port, RF link, an IR link, modem (telephone, cable, Ethernet, internet or other). In short, any technique for communicating information with system 10 is contemplated as the user interface.

In some embodiments, the present patent application describes a component within a noninvasive ventilator that derives, stores and analyses parameters derived from indices of the respiratory impedance that can monitor and alert a change or improvement in a person's pulmonary health. In some embodiments, not only can system 10 show the negative decline in symptoms or health but the trajectory of improvement in pulmonary function trending towards a patient's baseline to reflect recovery. Thus, having the ability to continuously monitor and track changes in a person's pulmonary impedance is advantageous and not currently available. In some embodiments, system 10 may be used in home healthcare solutions or systems. In some embodiments, system 10 may be used in home respiratory systems. In some embodiments, system 10 may be used for the COPD patients.

In some embodiments, system 10 may also include a communication interface that is configured to send the determined control signals to adjust the pressurized flow of breathable gas through an appropriate wireless communication method (e.g., Wi-Fi, Bluetooth, internet, etc.) to pressure regulator 14 or systems for further processing. In some embodiments, system 100 may include a recursive tuning subsystem that is configured to recursively tune its intelligent decision making subsystem using available data or information to provide better overall adjustment of the pressurized flow of breathable gas and/or better overall control of pressure regulator 14. In some embodiments, intelligent decision making subsystem, communication interface and recursive tuning subsystem may be part of computer system 19 (comprising server 20).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system configured to provide pressure therapy to a subject, the system comprising:
a pressure generator configured to provide a pressurized flow of breathable gas to an airway of the subject;
one or more sensors configured to generate output signals conveying information related to one or more parameters of the pressurized flow of breathable gas and respiration of the subject; and
one or more hardware processors operatively connected with the pressure generator and the one or more sensors, the one or more hardware processors configured by machine readable instructions to:
determine the one or more parameters of the pressurized flow of breathable gas and the respiration of the subject based on the information in the output signals;
determine one or more indices of respiratory impedance for the airway of the subject based on the determined one or more parameters;
determine a change in health status of the subject based on the one or more indices of respiratory impedance; and
generate an alert indicative of the change in health status for communication to the subject and/or a caregiver of the subject.

2. The system of claim 1, wherein the one or more hardware processors are configured such that the one or more indices of respiratory impedance comprise a respiratory resistance value, a respiratory impedance value, and a respiratory reactance value, and wherein the expiratory reactance value is determined based on the respiratory impedance value.

3. The system of claim 2, wherein the one or more hardware processors are configured such that the respiratory resistance value, the respiratory impedance value and the respiratory reactance value are determined for individual breaths in an ongoing manner during the pressure therapy.

4. The system of claim 3, wherein the one or more hardware processors are configured such that determining the one or more indices of respiratory impedance for the subject based on the determined one or more parameters comprises determining a breath to breath change in the expiratory reactance value, and wherein the one or more hardware processors are configured to determine the change in health status of the subject based on the breath to breath change in the expiratory reactance value to provide the pressure therapy to the subject.

5. The system of claim 4, wherein the one or more hardware processors are further configured to determine a threshold for the breath to breath change in the expiratory reactance value, and generate the alert indicative of the change in health status responsive to a breach of the threshold by the breath to breath change in the expiratory reactance value.

6. The system of claim 5, wherein the one or more hardware processors are further configured to determine the threshold for the breath to breath change in the expiratory reactance value based on a seasonality or a periodicity of the breath to breath change in the expiratory reactance value.

7. The system of claim 2, wherein the one or more hardware processors are configured such that the expiratory reactance value is a total airway expiratory reactance value, and wherein the total airway of the subject comprises a mouth, nasal passages, sinuses, pharynx, trachea, and lungs of the subject.

8. The system of claim 1, wherein the one or more hardware processors are further configured to control the pressure generator to adjust the pressurized flow of breathable gas based on the generated alert and the determined one or more indices of respiratory impedance to provide the pressure therapy to the subject.

9. A method for providing pressure therapy to a subject, the method being implemented by a computer system that comprises one or more hardware processors executing machine readable instructions that, when executed, perform the method, the method comprising:
obtaining, from one or more sensors, information related to one or more parameters of a pressurized flow of breathable gas to an airway of the subject and respiration of the subject;
determining, using the computer system, the one or more parameters of the pressurized flow of breathable gas and the respiration of the subject based on the obtained information;
determining, using the computer system, one or more indices of respiratory impedance for the airway of the subject based on the determined one or more parameters; and
determining, using the computer system, a change in health status of the subject based on the one or more indices of respiratory impedance; and
generating, using the computer system, an alert indicative of the change in health status for communication to the subject and/or a caregiver of the subject.

10. The method of claim 9, wherein the one or more indices of respiratory impedance comprise a respiratory resistance value, a respiratory impedance value, and a respiratory reactance value, and wherein the method comprises determining the expiratory reactance value based on the respiratory impedance value.

11. The system of claim 10, wherein the method comprises determining the respiratory resistance value, the respiratory impedance value and the respiratory reactance value for individual breaths in an ongoing manner during the pressure therapy.

12. The method of claim 11, wherein determining the one or more indices of respiratory impedance for the subject based on the determined one or more parameters comprises determining a breath to breath change in the expiratory reactance value, and wherein the method comprises determining the change in health status of the subject based on the breath to breath change in the expiratory reactance value to provide the pressure therapy to the subject.

13. The method of claim 12, wherein the method comprises determining a threshold for the breath to breath change in the expiratory reactance value, and generating the alert indicative of the change in health status responsive to a breach of the threshold by the breath to breath change in the expiratory reactance value.

14. The method of claim 13, wherein determining the threshold for the breath to breath change in the expiratory reactance value based on a seasonality or a periodicity of the breath to breath change in the expiratory reactance value.

15. A system providing pressure therapy to a subject, the system comprising:
a means for providing a pressurized flow of breathable gas to an airway of the subject;
a means for generating output signals conveying information related to one or more parameters of the pressurized flow of breathable gas and respiration of the subject; and
a means for executing machine-readable instructions with at least one hardware processor, wherein the machine-readable instructions comprising:
obtaining, from the means generating output signals, information related to one or more parameters of the pressurized flow of breathable gas and the respiration of the subject;
determining, using the means for executing, the one or more parameters of the pressurized flow of breathable gas and the respiration of the subject based on the obtained information;
determining, using the means for executing, one or more indices of respiratory impedance for the airway of the subject based on the determined one or more parameters;
determining, using the means for executing, a change in health status of the subject based on the one or more indices of respiratory impedance; and
generating, using the means for executing, an alert indicative of the change in health status for communication to the subject and/or a caregiver of the subject.
